(19) United States
(12) Patent Application Publication
(10) Pub. No.: US 2024/0150556 A1
(43) Pub. Date: May 9, 2024

(54) ECO-FRIENDLY STRUCTURE CONTAINING NATURAL FIBER AND REGENERATIVE FIBER

(71) Applicant: AMOREPACIFIC CORPORATION, Seoul (KR)

(72) Inventor: Jung Sun CHOI, Yongin-si (KR)

(73) Assignee: AMOREPACIFIC CORPORATION, Seoul (KR)

(21) Appl. No.: 18/485,188

(22) Filed: Oct. 11, 2023

(30) Foreign Application Priority Data
Oct. 14, 2022 (KR) 10-2022-0132484

(51) Int. Cl. C08L 1/02; A61K 8/02; A61K 8/73; A61K 8/81; A61Q 19/00; C08L 23/06

(52) U.S. Cl. CPC C08L 1/02; A61K 8/027; A61K 8/731; A61K 8/8111; A61Q 19/00; C08L 23/06; C08L 2205/16; C08L 2207/20

(57) ABSTRACT

Disclosed herein are an eco-friendly structured body including a natural fiber and a regenerative fiber, and a cosmetic product including the same, an eco-friendly structured body including a natural fiber and a regenerative fiber, according to one aspect of the present disclosure, has a weight ratio of the natural fiber to the regenerative fiber in a specific range, and an average length or an average thickness of the fibers, and the structured body includes the fiber as a material, but have a similar structural configuration with an elastic foam carrier, which is a filament trap structured body in which, when a cosmetic composition is carried in the structured body, a portion of the carried cosmetic composition is collected.

### CROSS REFERENCE TO RELATED APPLICATION

The present application claims priority to Korean Patent Application No. 10-2022-0132484, filed Oct. 14, 2022, the entire contents of which is incorporated herein for all purposes by this reference.

### BACKGROUND OF THE INVENTION

### Field of the Invention

The present specification relates to an eco-friendly structured body including a natural fiber and a regenerative fiber, and to cosmetic product including the same.

### Description of the Related Art

Conventionally, a liquid cosmetic composition is filled into a vacuum container, a pump container, or a glass container for distribution and storage, but there is a disadvantage of inconvenient portability. Therefore, as there is an increasing need to easily apply or modify makeup even when going out, a cosmetic composition carrier that is capable of uniformly carrying a liquid cosmetic composition for a long period of time has emerged. However, the carrier of cosmetic composition made of foam is a petroleum-derived raw material, and there was a problem of insufficient environmental contribution after disposal.

In consideration of these environmental problems, an eco-friendly structured body using an environmentally beneficial material has been researched. However, even if the eco-friendly material is used, the carrier that carries the cosmetic composition needs to have the stability to maintain the structure thereof even when stored and distributed for a long time. In addition, even if the carrier carries a cosmetic composition in which oil-based and water-based components are mixed, the oil-based component should not be separated, and the structured body should have enough elasticity to allow the cosmetic composition carried inside to be uniformly discharged in an appropriate amount by pressing the structured body. In addition, the carrier should also have the property of being naturally decomposed after disposal as an eco-friendly material.

Therefore, the present inventors have researched a structured body that has the property of being usable for carrying the cosmetic composition, but also has the property of being eco-friendly by being naturally decomposed after disposal, and have developed an eco-friendly structured body that has all of the above properties by mixing a natural fiber and a regenerative fiber.

### SUMMARY OF THE INVENTION

The present inventors have conducted research on a structured body that carries a liquid phase composition and maintains the shape thereof during the period of use, but thereafter naturally is decomposed and has eco-friendly properties.

As a result of conducting the research, it was confirmed that the structured body composed of a mixture of a natural fiber and a regenerative fiber maintains the shape thereof during the period of use and has elasticity without separation of an oil phase component of a cosmetic composition carried inside, thereby providing an excellent sense of use, but also has properties that are beneficial to the environment by naturally being decomposed thereafter, thus completing the present invention.

Therefore, according to one aspect, an object of the present disclosure is directed to providing an eco-friendly structured body including a natural fiber and a regenerative fiber.

According to another aspect, an object of the present disclosure is directed to providing a cosmetic product including the eco-friendly structured body, and a cosmetic composition carried in the eco-friendly structured body.

It is one aspect of the present disclosure to provide an eco-friendly structured body including a natural fiber and a regenerative fiber, and having one or more of characteristics of (A) to (C), in which: (A) an average length of one or more of those selected from the group consisting of the natural fiber and the regenerative fiber is 40 to 80 mm; (B) a weight ratio of the natural fiber to the regenerative fiber is 85:15 to 20:80; and (C) an average thickness of one or more of those selected from the group consisting of the natural fiber and the regenerative fiber is 15 to 25 μm.

It is another aspect of the present disclosure to provide a cosmetic product including the eco-friendly structured body, and a cosmetic composition carried in the eco-friendly structured body.

An eco-friendly structured body including a natural fiber and a regenerative fiber, according to one aspect of the present disclosure, has a weight ratio of the natural fiber to the regenerative fiber in a specific range, and an average length or an average thickness of the fibers, and the structured body includes the fiber as a material, but have a similar structure with an elastic foam carrier, which is a filament trap structured body in which, when a cosmetic composition is carried in the structured body, a portion of the carried cosmetic composition is collected. Therefore, an oil phase component of the cosmetic composition is not separated, and the structured body has the stability to maintain the structure thereof during the period of distribution and storage after being manufactured, and when the structured body is pressed, the carried cosmetic composition is discharged in a finely diffused manner, thereby having an excellent cosmetic effect, but also having the environmentally beneficial characteristic of being naturally decomposed after use.

### DETAILED DESCRIPTION OF THE INVENTION

In one aspect of the present disclosure, a “carrier” means one capable of carrying any material or component, such as a composition, and may also be referred to as a “support,” “impregnation agent,” or “medium.” In addition, the “carrier” may be one that is used to discharge a material carried therein into a separate application tool. The composition carried in the carrier may be transferred to the skin, for example, by hand or by means of an application means (also referred to as an application tool or an applicator) such as a puff, tip, brush, etc.

In one aspect of this disclosure, “carrying capacity” means an ability to carry and retain any material or component. The “carrying capacity” required of the carrier is to homogeneously carry the composition for a long period of time, which is distinct from temporary taking up of the material by the application tool, etc.

In one aspect of the present disclosure, “filling capability” or “filling ability” refers to a capability of a structured body (carrier) to be filled with a cosmetic composition, which can be represented by a time taken to fill the structured body (carrier) with a certain amount of cosmetic composition. In one aspect of the present disclosure, the measurement of “filling capability” or “filling ability” means a time taken to manually fill a structured body (carrier) having a dimension of 56 mm in diameter by 7.0 mm in height with 5,000 to 20,000 cps of a cosmetic composition.

In one aspect of the present disclosure, “discharge capability” or “discharge ability” means the amount of cosmetic composition that is discharged when the cosmetic composition is taken by the application tool from the carrier carrying the cosmetic composition, and it is preferred that a suitable amount of cosmetic composition, neither too much nor too little, is discharged. In one aspect of the present disclosure, the measurement of “discharge capacity” or “discharge ability” is a measurement value taken when a weight (force) of 1 to 2 kg is pressed against a surface of the structured body (carrier) having a dimension of 56 mm in diameter by 7.0 mm in height filled with a cosmetic composition of 5,000 to 20,000 cps.

In one aspect of the present disclosure, “durability” means a degree to which, when the structured body (carrier) is carried in a cosmetic composition and left for a certain period of time at a certain temperature, some volume of a total volume of the structured body (carrier) is not reduced, such as by melting or tearing of the structured body (carrier), and/or a degree to which the structured body (carrier) withstands repeated pressure from an application tool when the cosmetic composition is taken from the structured body (carrier) by the application tool during use.

Hereinafter, the present disclosure will be described in detail.

In one aspect, the present disclosure includes a natural fiber and a regenerative fiber, and provides an eco-friendly structured body having one or more of the following characteristics: (A) an average length of one or more selected from the group consisting of the natural fiber and the regenerative fiber is 40 to 80 mm; (B) a weight ratio of the natural fiber to the regenerative fiber is 85:15 to 20:80; and (C) an average thickness of one or more selected from the group consisting of the natural fiber and the regenerative fiber is 15 to 25 μm.

An eco-friendly structured body according to one aspect of the present disclosure may include a natural fiber and a regenerative fiber.

According to one embodiment of the present disclosure, a structured body including a natural fiber and a regenerative fiber has a filament trap structure in which a portion of the cosmetic composition carried in the structured body is collected, such that an oil phase and an aqueous phase of the cosmetic composition are not separated, and the carried cosmetic composition is discharged in a diffusive manner when pressure is applied to the structured body. Therefore, it was confirmed that the discharged cosmetic composition has an excellent cosmetic effect because a cosmetic film is thinner and a sense of adhesion is improved when the cosmetic composition is applied to the skin (see Experimental example 1 and Table 1).

In one aspect of the present disclosure, a “natural fiber” is a naturally produced fiber. The natural fiber may be one or more of those selected from the group consisting of a cellulose-based fiber and a protein-based fiber, and more particularly one or more of those selected from the group consisting of cotton, cellulose, and a polylactic acid (PLA) fiber, and more particularly cellulose, but is not limited thereto, as long as the natural fiber carries a cosmetic composition and the property of naturally being decomposed when mixed with a regenerative fiber.

In one aspect of the present disclosure, a “regenerative fiber” is a type of fiber in which a natural or artificial fibrous polymeric material is brought to a homogeneous state by dissolving, melting, or the like, and is formed into a fiber again. The regenerative fiber may be one or more of those selected from the group consisting of rayon, polyester (PET), polyethylene (PE), polypropylene (PP), thermoplastic elastomer (TPE), thermoplastic polyurethane (TPU), natural pulp fiber, nylon, and acetate, and more specifically, may be one or more of those selected from the group consisting of rayon, polyester (PET), polypropylene (PP), and nylon, and more specifically, may be polyester, but is not limited thereto, as long as the natural fiber carries a cosmetic composition and the property of being naturally decomposed when mixed with a regenerative fiber.

An average length of one or more of those selected from the group consisting of the natural fiber and the regenerative fiber, according to one aspect of the present disclosure, may be 40 to 80 mm. Specifically, the average length of one or more of those selected from the group consisting of the natural fiber and the regenerative fiber may be 40 mm or more, 42 mm or more, 44 mm or more, 46 mm or more, 48 mm or more, 50 mm or more, 52 mm or more, 54 mm or more, 56 mm or more, 58 mm or more, 60 mm or more, 62 mm or more, 64 mm or more, 66 mm or more, 68 mm or more, 70 mm or more, 72 mm or more, 74 mm or more, 76 mm or more, or 78 mm or more, and may be 80 mm or less, 78 mm or less, 76 mm or less, 74 mm or less, 72 mm or less, 70 mm or less, 68 mm or less, 66 mm or less, 64 mm or less, 62 mm or less, 60 mm or less, 58 mm or less, 56 mm or less, 54 mm or less, 52 mm or less, 50 mm or less, 48 mm or less, 46 mm or less, 44 mm or less, or 42 mm or less, but is not limited thereto. When the average length of one or more of those selected from the group consisting of the natural fiber and the regenerative fiber is less than 40 mm, fluff is formed on a surface of the fiber, which reduces the stability. In addition, when the average length of one or more of those selected from the group consisting of the natural fiber and the regenerative fiber is greater than 80 mm, the oil phase component of the cosmetic composition carried inside the structured body (carrier) moves along the fiber, causing the oil phase component to separate and not adequately carry the liquid phase of cosmetic composition.

According to an embodiment of the present disclosure, when the average length of one or more of those selected from the group consisting of the natural fiber and the regenerative fiber is 40 to 80 mm, it was confirmed that the oil phase component of the cosmetic composition is not separated, so that the cosmetic composition is stably contained in the structured body (carrier), while having excellent stability by not forming fluff even after use for a long period of time (Example 5 and Table 6).

A weight ratio of the natural fiber to the regenerative fiber according to one aspect of the present disclosure may be 85:15 to 20:80. Specifically, the weight ratio of the natural fiber to the regenerative fiber may be 85:15 or less, 80:20 or less, 75:25 or less, 70:30 or less, 65:35 or less, 60:40 or less, 55:45 or less, 50:50 or less, 45:55 or less, 40:60 or less, 35:65 or less, 30:70 or less, or 25:75 or less, and may be 20:80 or more, 25:75 or more, 30:70 or more, 35:65 or more, 40:60 or more, 45:55 or more, 50:50 or more, 55:45 or more, 60:40 or more, 65:35 or more, 70:30 or more, 75:25 or more, or 80:20 or more, but is not limited thereto. When the weight ratio of the natural fiber to the regenerative fiber in the structured body exceeds 85:15, the durability of the structured body decreases when carrying the cosmetic composition, which leads a structural collapse during the period of distribution or use, and the structured body is not suitable for use as a carrier that carries the cosmetic composition. In addition, when the weight ratio of the natural fiber to the regenerative fiber in the structured body is less than 20:80, there is a possibility of contaminating or destroying the nature environment since the structured body does not decompose naturally even after the period of use after the cosmetic composition is carried in the structured body.

According to an embodiment of the present disclosure, when the weight ratio of the natural fiber to the regenerative fiber is 85:15 to 20:80, the structure is maintained even when stored at a high temperature for 3 weeks, and a portion of the total volume of the carrier is decomposed after 5 weeks, which is expected to be naturally hydrolyzed. Therefore, it is confirmed that the eco-friendly structured body according to one aspect of the present disclosure has both durability and eco-friendly properties (Experimental example 3 and Table 4).

An average thickness of one or more of those selected from the group consisting of the natural fiber and the regenerative fiber, according to one aspect of the present disclosure, may be 15 to 25 μm. Specifically, the average thickness of one or more of those selected from the group consisting of the natural fiber and the regenerative fiber may be 15 μm or more, 16 μm or more, 17 μm or more, 18 μm or more, 19 μm or more, 20 μm or more, 21 μm or more, 22 μm or more, 23 μm or more, or 24 μm or more, and may be 25 μm or less, 24 μm or less, 23 μm or less, 22 μm or less, 21 μm or less, 20 μm or less, 19 μm or less, 18 μm or less, 17 μm or less, or 16 μm or less, but is not limited thereto. When the average thickness of one or more of those selected from the group consisting of the natural fiber and the regenerative fiber is less than 15 μm, the density of the structured body (carrier) is high, resulting in a dense fiber tissue, and a very small amount of the cosmetic composition is discharged, thereby reducing the cosmetic effect. In addition, when the average thickness of one or more of those selected from the group consisting of the natural fiber and the regenerative fiber is greater than 25 μm, the density of the structured body (carrier) is low, so that the fiber tissue is rough and coarse, which causes a large amount of cosmetic composition to be discharged, thus resulting in clumping of the cosmetic film and thickening of the makeup and reducing the sense of use.

According to one embodiment of the present disclosure, when the average thickness of one or more of those selected from the group consisting of the natural fiber and the regenerative fiber is 15 to 25 μm, the tissue consisting of the carrier becomes fine. Accordingly, an appropriate amount of cosmetic composition is finely discharged, and a thin cosmetic film with high coverage can be expressed, which has an effect of improving a sense of use (Example 4 and Table 5).

The structured body according to one aspect of the present disclosure may be a structure in which the natural fiber and the regenerative fiber are twisted.

The structured body according to one aspect of the present disclosure may have unevenness, bumps, or twists formed on at least a portion of the surface of one or more of those selected from the group consisting of the natural fiber and the regenerative fiber. More specifically, the structured body may have unevenness, bumps, or twists formed on more than 0% but less than or equal to 100% of a total area of the surface of one or more of those selected from the group consisting of the natural fiber and the regenerative fiber, more specifically, the structured body may have unevenness, bumps, or twists formed on more than 0%, 1% or more, 10% or more, 20% or more, 30% or more, 40% or more, 50% or more, 60% or more, 70% or more, 80% or more, or 90% or more of the total area of the surface of one or more of those selected from the group consisting of the natural fiber and the regenerative fiber, and may have unevenness, bumps, or twists formed on 100% or less, 99% or less, 90% or less, 80% or less, 70% or less, 60% or less, 50% or less, 40% or less, 30% or less, 20% or less, or 10% or less thereof. According to an embodiment of the present disclosure, it was confirmed that when one or more surfaces selected from the group consisting of the natural fiber and the regenerative fiber are linear with no unevenness, bumps, or twists, the surface has no elasticity, whereas when unevenness, bumps, or twists are formed, the elasticity is increased, resulting in uniform discharge of the cosmetic composition carried therein in a more suitable amount, and a better sense of use (Example 6 and Table 7).

The carrier according to one aspect of the present disclosure may include a fiber having a ratio of the longest diameter to the shortest diameter of one or more cross-sections selected from the group consisting of the natural fiber and the regenerative fiber exceeding 1. The one or more of those selected from the group consisting of the natural fiber and the regenerative fiber according to one aspect of the present disclosure may be in a flattened form. According to an embodiment of the present disclosure, when the fiber included in the carrier is cotton having a cross-section of a circular or square shape such that a length of the shortest diameter of the cross-section and a length of the longest diameter of the cross-section are similar, a suitable amount of the cosmetic composition carried in the carrier may not be discharged, but when the ratio of the longest diameter to the shortest diameter of the cross-section of the fiber exceeds 1, a suitable amount of the cosmetic composition is uniformly discharged (Experimental example 2 and Table 3).

The density of the structured body according to one aspect of the present disclosure may be 0.015 to 0.032 g/cm³. Specifically, the density of the structured body may be 0.015 g/cm³ or more, 0.016 g/cm³ or more, 0.018 g/cm³ or more, 0.02 g/cm³ or more, 0.022 g/cm³ or more, 0.024 g/cm³ or more, 0.026 g/cm³ or more, 0.028 g/cm³ or more or 0.03 g/cm³ or more, and may be 0.032 g/cm³ or less, 0.03 g/cm³ or less, 0.028 g/cm³ or less, 0.026 g/cm³ or less, 0.024 g/cm³ or less, 0.022 g/cm³ or less, 0.02 g/cm³ or less, 0.018 g/cm³ or less or 0.016 g/cm³ or less, but is not limited thereto. When the density of the structured body is less than 0.015 g/cm³, the density is too low to carry the cosmetic composition, and the fiber tissue is rough and coarse, and the amount of cosmetic composition discharged is large, resulting in clumping of the cosmetic film and thickening of the makeup, resulting in a poor sense of use. In addition, when the density of the structured body exceeds 0.032 g/cm³, the density of the structured body is too high to carry the cosmetic composition, and the fiber tissue is dense, resulting in a very small amount of cosmetic composition being discharged, which reduces the cosmetic effect.

The structured body according to one aspect of the present disclosure may have a thickness of 0.1 to 10 mm. Specifically, the thickness of the structured body may be 0.1 mm or more, 0.5 mm or more, 1 mm or more, 1.5 mm or more, 2 mm or more, 2.5 mm or more, 3 mm or more, 3.5 mm or more, 4 mm or more, 4.5 mm or more, 5 mm or more, 5.5 mm or more, 6 mm or more, 6.5 mm or more, 7 mm or more, 7.5 mm or more, 8 mm or more, 8.5 mm or more, 9 mm or more or 9.5 mm or more, and may be 10 mm or less, 9.5 mm or less, 9 mm or less, 8.5 mm or less, 8 mm or less, 7.5 mm or less, 7 mm or less, 6.5 mm or less, 6 mm or less, 5.5 mm or less, 5 mm or less, 4.5 mm or less, 4 mm or less, 3.5 mm or less, 3 mm or less, 2.5 mm or less, 2 mm or less, 1.5 mm or less, 1 mm or less or 0.5 mm or less, but is not limited thereto. When the thickness of the structured body is less than 0.1 mm, the amount of cosmetic composition carried is too small, and when the thickness exceeds 10 mm, it is difficult to discharge the cosmetic composition without a residual amount when used.

The structured body according to one aspect of the present disclosure may be for carrying a cosmetic composition.

The structured body according to one aspect of the present disclosure may have a structure that includes a plurality of storage spaces in which a portion of the carried cosmetic composition is collected, and more specifically, the structure may be a filament trap structure. In general, when the carrier in which the cosmetic composition is carried is made of a fabric material, the carrier has a web structure in which the fibers are tangled, and the carried cosmetic composition flows along the fibers, causing the oil phase component to be separated, which leads to instability. In contrast, since the structured body (carrier) according to one aspect of the present disclosure has a plurality of storage spaces in which a portion of the carried cosmetic composition is collected, the flow of the oil phase component of the cosmetic composition along the fibers is interrupted so that separation of the oil phase component does not occur, and due to this fiber trap structure, the carried cosmetic composition is discharged in a finely diffused manner when the structured body (carrier) is pressed, which has an excellent effect.

The cosmetic composition according to one aspect of the present disclosure may be a liquid composition, and may include a solution, emulsion, gel, cream, or suspension. It is more difficult to carry and store the cosmetic composition in a liquid phase than in a solid phase, but when the structured body (carrier) according to one aspect of the present disclosure is used, the liquid phase or cream phase cosmetic composition may be stably and safely stored and carried.

The cosmetic composition according to one aspect of the present disclosure may be a type of emulsion, specifically a type of water-in-oil (W/O) or oil-in-water (O/W), or may be a type of dispersion, specifically a type of oil-dispersion or water-dispersion.

The cosmetic composition according to one aspect of the present disclosure may have a viscosity of 5,000 to 20,000 centi poise (cps). The viscosity may be measured by filling a 40 ml stability container with the contents, then storing it in a 30° C. chamber for at least one day, and then operating it on a Brookfield LVII viscometer using spindle 4 at 30 rpm for one minute. Specifically, the viscosity of the cosmetic composition may be 5,000 cps or more, 5,500 cps or more, 6,000 cps or more, 6,500 cps or more, 7,000 cps or more, 7,500 cps or more, 8,000 cps or more, 8,500 cps or more, 9,000 cps or more, 9,500 cps or more, 10,000 cps or more, 11,000 cps or more, 12,000 cps or more, 13,000 cps or more, 14,000 cps or more, 15,000 cps or more, 16,000 cps or more, 17,000 cps or more, 18,000 cps or more or 19,000 cps or more, and may be 20,000 cps or less, 19,000 cps or less, 18,000 cps or less, 17,000 cps or less, 16,000 cps or less, 15,000 cps or less, 14,000 cps or less, 13,000 cps or less, 12,000 cps or less, 11,000 cps or less, 10,000 cps or less, 9,500 cps or less, 9,000 cps or less, 8,500 cps or less, 8,000 cps or less, 7,500 cps or less, 7,000 cps or less, 6,500 cps or less, 6,000 cps or less or 5,500 cps or less, but is not limited thereto. The structured body according to one aspect of the present disclosure is capable of carrying the cosmetic composition of low viscosity therein and still uniformly discharging a suitable amount of the cosmetic composition without separation of the oil phase component of the cosmetic composition, having elasticity, and being structurally maintained during the period of use.

The cosmetic composition according to one aspect of the present disclosure may be selected from the group consisting of a cosmetic composition for skin care, a cosmetic composition for makeup, and a UV protection agent, and may be specifically formulated as, but is not limited to, a twin cake, makeup primer, makeup base, foundation, concealer, lipstick, lip gloss, powder, lip liner, eyebrow, eyeliner, eyeshadow, blush, UV protection agent, lotion, cream, or essence.

The structured body according to one aspect of the present disclosure may be for use with the cosmetic composition carried in the structured body applied to an application tool. The application tool is not limited as long as it is a separate tool that enables the cosmetic composition to be taken from the structured body (carrier) in which the cosmetic composition is carried. For example, the application tool may be a hand, a puff, a tip, a brush, or the like, and may also be referred to as an application means or an applicator. That is, the structured body according to one aspect of the present disclosure is a carrier in which the cosmetic composition is carried, rather than an application tool, and a separate application tool needs to be used to take the cosmetic composition from the carrier.

The structured body according to one aspect of the present disclosure may be a multilayer structure having a plurality of layers.

The structured body according to one aspect of the present disclosure may configured to have at least one surface of a bottom surface (a surface opposite to a surface in contact with a hand or application tool) and a lateral surface of the structured body with a structure that impedes the flow of the cosmetic composition, specifically, the structure may be a coating film, and the structure that impedes the flow of the cosmetic composition may prevent leakage of the cosmetic composition downwardly or laterally in a state where the cosmetic composition is carried in the structured body. As described above, when the lateral surface or the bottom surface of the structured body is configured with the structure that impedes the flow of the cosmetic composition, it is possible to reduce the leakage of the cosmetic composition out of the structured body by the cosmetic composition moving to the lateral surface or the bottom surface during use, thereby reducing the loss of the cosmetic composition, thereby increasing the period of use, and improving the convenience.

In another aspect, the present disclosure provides cosmetic product including the eco-friendly structured body, and a cosmetic composition carried in the eco-friendly structured body. The description of the eco-friendly structured body and cosmetic composition is as described above.

The cosmetic product according to one aspect of the present disclosure may further include an application tool. The application tool is not limited as long as it is a separate tool that enables the cosmetic composition to be taken from the structured body in which the cosmetic composition is carried. For example, the application tool may be a hand, a puff, a tip, a brush, or the like, and may also be referred to as an application means or an applicator. That is, the structured body according to one aspect of the present disclosure is a carrier in which the cosmetic composition is carried, rather than an application tool, and a separate application tool needs to be used to take the cosmetic composition from the carrier.

Hereinafter, the configuration and effect of the present disclosure will be described in more detail with reference to examples and experimental examples. However, the following examples, manufacturing examples, and experimental examples are provided for illustrative purposes only to assist in understanding the present disclosure and are not intended to limit the scope and range of the present disclosure.

### [Experimental Example 1] Confirmation of Difference and Stability in Cosmetic Composition Discharge Method According to Material of Structured Body

The structured body for carrying the cosmetic composition is a carrier that carries the cosmetic composition therein and has a structure such that the cosmetic composition is discharged when the structured body is pressed. The structured body has a different structure depending on a material of the structured body, and accordingly, the cosmetic composition discharge method varies. The present inventors have carried out the following experiments to confirm with which method the cosmetic composition is discharged depending on the material of the structured body, and to confirm that the structural stability is maintained when the cosmetic composition having the composition of Table 1 below, including the oil phase component, is carried therein, and the results are shown in FIG. 2. In this case, the manufactured structured body has dimensions of 56 mm in diameter by 7.0 mm in height.

TABLE 1
Unit: wt %

| | | Manufacturing |
|---|---|---|
| Classification | Component | Example 2 |
| Oil phase | Cetylpeg/PPG-10/1 dimethicone | 2.00 |
| component | Phenyl trimethicone | 12.00 |
| | Cyclopentasiloxane | 13.00 |
| | Ethylhexyl methoxycinnamate | 6.50 |
| | Caprylic/Capric triglyceride | 3.00 |
| Thickening agent | Disteardimonium hectorite | 1.20 |
| Inorganic Powder | Titanium oxide | 15.00 |
| Inorganic Powder | Iron oxide | 2.00 |
| Water phase | Purified water | To 100 |
| component | Disodium-EDTA | 0.05 |
| | Sodium chloride (NaCl) | 1.00 |
| | Glycerin | 5.00 |
| | Ethylhexyl Glycerin | Suitable Amount |

TABLE 2

| | Comparative | Comparative | |
|---|---|---|---|
| | example 1-1 | example 1-2 | Example 1 |
| Material of | Polyurethane | Polyethylene | Rayon/ |
| structured | | (PE)/ | Regenerative |
| body | | Polypropylene | Polyethylene |
| | | (PP) | (PE) |
| Structure of | Polygonal | Web | Fiber trap |
| structured | structure | structure | structure |
| body (Liquid phase content impregnation structure) | [symbol] | [symbol] | [symbol] |
| Cosmetic | Discharge as | Discharge | Diffusion |
| composition | droplet | in bulk | discharge |
| discharge | | | |
| method | | | |
| Stability | Good | Oil phase | Good |
| | | component | |
| | | separation | |

Comparative examples 1-1 above are a conventional foam carrier, which has a polygonal, for example, a pentagonal structure including a plurality of pores therein. The foam carrier carries the cosmetic composition in pores having a polygonal structure, and when the carrier is pressed, the carried cosmetic composition is discharged in the form of a droplet, and the oil phase and the aqueous phase of the cosmetic composition are stable with no separation. However, the foam carrier of Comparative example 1-1 above is manufactured with a petroleum-derived raw material as a material, which has insufficient environmental contribution.

The carrier of Comparative example 1-2 above is a structured body (carrier) of a web structure made of PE/PP, which is a synthetic fiber, and the synthetic fiber is a long fiber, from which the carried cosmetic composition is discharged in bulk. In particular, when the structured body of Comparative example 1-2 is pressed, the carried cosmetic composition is just discharged in bulk instantaneously, and the discharge amount of the cosmetic composition decreases rapidly during the period of use, which causes inconvenience. Furthermore, the structured body of Comparative example 1-2 is unstable because the oil phase component of the cosmetic composition flows along the fibers forming the structured body, which causes the oil phase component in the cosmetic composition to be separated. A problem with the structured body of Comparative example 1-2 above is that the structured body (carrier) is made up of fibers and has a web structure in which the fibers are entangled, resulting in large pores in which the cosmetic composition carried therein can be stored, and it is expected that the oil component of the cosmetic composition is separated by the fibers.

In contrast, Example 1 above is an eco-friendly structured body according to one aspect of the present disclosure that is made of a material that is a mixture of the natural fiber and the regenerative fiber. The structured body of embodiment 1 includes short fibers of a short length, such that the flow of the oil phase component of the cosmetic composition carried within the structured body along the fibers is interrupted and the structured body has a filament trap structure in which a portion of the cosmetic composition is collected, thereby preventing separation of the oil and water phases of the cosmetic composition. In addition, due to the length of these fibers and the fiber trap structure, when the structured body is pressed, the carried cosmetic composition is discharged in a finely diffused manner compared to the structured body of Comparative example 1-2 above, and when the discharged cosmetic composition is applied to the skin, the cosmetic film is thinner and a sense of adhesion is improved, resulting in an excellent cosmetic effect.

Accordingly, the eco-friendly structured body including the natural fiber and the regenerative fiber according to one aspect of the present disclosure includes a fiber as a material but has a structure such as an elastic foam carrier. Therefore, when the structured body is pressed, the cosmetic composition is discharged in a diffused manner with excellent stability. As a result, it was confirmed that the structured body has an excellent cosmetic effect.

### [Experimental Example 2] Comparison of Durability and Discharge Capability of Structured Body According to Fiber Type

From Experimental example 1 above, it was confirmed that the structured body including the natural fiber and the regenerative fiber has excellent stability and a sense of use. The eco-friendly structured body according to one aspect of the present disclosure is capable of carrying the cosmetic composition and releasing the cosmetic composition with elasticity. In addition, the eco-friendly structured body should have the durability to carry the cosmetic composition and maintain a state of being usable during the period of use. Therefore, the durability and discharge capability of the structured body were compared for each type of natural and regenerative fiber to be mixed, and the results are shown in Table 3. In this case, the manufactured structured body has a dimension of 56 mm in diameter by 7.0 mm in height, and a method of evaluating the durability and discharge capability is as follows.

### Method of Evaluating Durability

When the structured body composed of the fibers of Table 3 below containing 15 grams of the cosmetic composition of Table 1 above was stored at a high temperature of 70° C. for three weeks, the volume of the structured body was compared, and as a result of the comparison, it was evaluated as poor when a portion of the structured body was decomposed, and excellent when the structured body was not decomposed and maintained the shape thereof.

### Method of Evaluating Discharge Capability

The structured body composed of the fibers in Table 3 below was filled with 15 grams of the cosmetic composition in Table 1 above and evaluated whether the suitable amount of the cosmetic composition carried in the structured body was uniformly discharged in a single application using a puff or hand from each structured body. In Table 3 below, Δ means that too much or too little cosmetic composition is discharged or not uniformly discharged, ○ means that a moderate amount of cosmetic composition is uniformly discharged, and ⊚ means that a very moderate amount of cosmetic composition is uniformly discharged for a long time.

TABLE 3

| | Type of natural fiber | | | Type of regenerative fiber | | | | |
|---|---|---|---|---|---|---|---|---|
| | Cotton | Cellulose | PLA | Rayon | PET | PP | NYLON6 | Polyethylene |
| Durability | Excellent | Excellent | Poor | Excellent | Excellent | Excellent | Excellent | Excellent |
| Discharge | Δ | ◯ | ◯ | ◯ | ⊚ | ◯ | ◯ | Δ |
| capability | | | | | | | | |

As shown in Table 3, it was confirmed that the stability and discharge capability of the structured body varies depending on the type of natural and regenerative fibers that are mixed. In this case, it is preferable that the discharge capability is not too little and not too much, so at the beginning of use, the extent of discharge of approximately 0.3 to 0.5 grams per payoff was determined to be a suitable amount. When the discharge capability is 0.3 g or less, the application amount is small, which reduces the cosmetic effect, and when the discharge capability is 0.5 g or more, clumping phenomenon occurs, which causes uneven application.

### [Experimental Example 3] Comparison of Durability and Degree of Decomposition According to Weight Ratio of Natural Fiber to Regenerative Fiber

The eco-friendly structured body according to one aspect of the present disclosure should remain usable while carrying the cosmetic composition during the period of use, but thereafter be naturally decomposed and have environmentally beneficial properties. Therefore, the durability and degree of decomposition of the natural fiber and regenerative fiber were compared by weight ratio, and the results are shown in Table 4. In this case, the structured body manufactured using cellulose as the natural fiber and PET as the regenerative fiber was evaluated with 15 grams of the cosmetic composition of Example 1 above.

### Method of Evaluating Durability

When the structured body maintains the shape thereof even when stored at high temperature (70° C.) for three weeks, it is determined that the structured body maintains a stable shape during the period when a user uses the structured body, and the method of evaluating the durability is as described in Experimental example 2 above.

### Method of Evaluating Degree of Decomposition

When the structured body was stored at a high temperature (70° C.), the change in volume was measured after 3 weeks or more (5 weeks has passed in Table 4 below), and when the volume decreased, it was evaluated as being decomposed, and when the volume did not decrease, it was evaluated as not being decomposed (stable).

TABLE 4

| | Compar- | Compar- | Compar- | Compar- | | | |
|---|---|---|---|---|---|---|---|
| | ative | ative | ative | ative | | | |
| | exam- | exam- | exam- | exam- | Exam- | Exam- | Exam- |
| | ple | ple | ple | ple | ple | ple | ple |
| | 2-1 | 2-2 | 2-3 | 2-4 | 2-1 | 2-2 | 2-3 |
| Natural | 100 | 96 | 95 | 90 | 85 | 80 | 70 |
| fiber | | | | | | | |
| (Wt %) | | | | | | | |
| Regen- | 0 | 4 | 5 | 10 | 15 | 20 | 30 |
| erative | | | | | | | |
| fiber | | | | | | | |
| (Wt %) | | | | | | | |
| Durability | Unstable | Unstable | Unstable | Unstable | Stable | Stable | Stable |
| Degree | Decomp- | Decomp- | Decomp- | Decomp- | Decomp- | Decomp- | Decomp- |
| of | osition | osition | osition | osition | osition | osition | osition |
| decomp- | | | | | | | |
| osition | | | | | | | |
| | | | | | | Compar- | Compar- |
| | | | | | | ative | ative |
| | Exam- | Exam- | Exam- | Exam- | Exam- | exam- | exam- |
| | ple | ple | ple | ple | ple | ple | ple |
| | 2-4 | 2-5 | 2-6 | 2-7 | 2-8 | 2-5 | 2-6 |
| Natural | 50 | 50 | 40 | 30 | 20 | 10 | 5 |
| fiber | | | | | | | |
| (Wt %) | | | | | | | |
| Regen- | 40 | 50 | 60 | 70 | 80 | 90 | 95 |
| erative | | | | | | | |
| fiber | | | | | | | |
| (Wt %) | | | | | | | |
| Durability | Stable | Stable | Stable | Stable | Stable | Stable | Stable |
| Degree | Decomp- | Decomp- | Decomp- | Decomp- | Decomp- | Stable | Stable |
| of | osition | osition | osition | osition | osition | | |
| decomp- | | | | | | | |
| osition | | | | | | | |

As shown in Table 4 above, when the weight ratio of the natural fiber to the regenerative fiber was 100:0 to 90:10 (Comparative examples 2-1 to 2-4), the structured body was unstable and collapsed in structure when stored at a high temperature for three weeks, and was not suitable for use as a carrier for the cosmetic composition. In addition, when the weight ratio of the natural fiber to the regenerative fiber was 10:90 and 5:96 (Comparative examples 2-5 and 2-6), the structured body was not decomposed naturally even when stored at a high temperature for 5 weeks, and therefore did not have the eco-friendly properties.

In contrast, when the weight ratio of the natural fiber to the regenerative fiber is 85:15 to 20:80 (Examples 2-1 to 2-8), the structure is maintained even when stored at a high temperature for 3 weeks, and a portion of the total volume of the structured body is decomposed after 5 weeks, which is expected to be naturally hydrolyzed. Therefore, it is confirmed that the eco-friendly structured body according to one aspect of the present disclosure has both durability and eco-friendly properties.

### [Example 4] Comparison of Sense of Use According to Fiber Thickness

The sense of use was evaluated according to the thickness of the natural fiber and the regenerative fiber constituting the eco-friendly structured body according to one aspect of the present disclosure. In this case, the structured body used cellulose as the natural fiber and PET as the regenerative fiber, and the weight ratio of the natural fiber to the regenerative fiber was approximately 40:60, and 15 grams of the cosmetic composition of Example 1 above, which differed only in the average thickness of the fibers was carried. In this case, the manufactured structured body has dimensions of 56 mm in diameter by 7.0 mm in height.

For the sense of use, 15 users who use makeup products were asked to use the structured body with different average fiber thicknesses and the results were evaluated.

TABLE 5

| | Compar- | Compar- | | | | Compar- | Compar- |
|---|---|---|---|---|---|---|---|
| | ative | ative | | | | ative | ative |
| | exam- | exam- | | | | exam- | exam- |
| | ple | ple | Example | Example | Example | ple | ple |
| | 3-1 | 3-2 | 3-1 | 3-2 | 3-3 | 3-3 | 3-4 |
| Fiber | 5 | 10 | 15 | 20 | 25 | 30 | 35 |
| thickness | | | | | | | |
| (μm) | | | | | | | |
| Sense of | Insufficient | Insufficient | Fine | Fise | Fine | Excess | Excess |
| use | discharge | discharge | discharge | discharge | discharge | discharge | discharge |
| | | | (Moderate | (Moderate | (Moderate | | |
| | | | amount | amount | amount | | |
| | | | discharge) | discharge) | discharge) | | |

As shown in Table 5 above, when the average thickness of the fibers constituting the structured body was less than 15 μm (Comparative examples 3-1 and 3-2), a very small amount of cosmetic composition was discharged because of the dense fiber tissue due to the high density of the structured body, and the cosmetic effect was reduced. In addition, when the average thickness of the fibers was greater than 30 μm (Comparative examples 3-3 and 3-4), the fiber tissue of the structured body was rough and coarse and the amount of cosmetic composition discharged was large, causing the cosmetic film to clump and the makeup to be thicker and the sense of use was poor.

In contrast, when the average thickness of the fibers is 15 to 25 μm (Examples 3-1 to 3-3), the tissues forming the structured body are fine, so that the suitable amount of cosmetic composition can be finely discharged, and it was confirmed that a thin cosmetic film with high coverage can be expressed, resulting in an excellent sense of use.

### [Example 5] Stability Evaluation According to Fiber Length

The stability according to lengths of the natural fiber and the regenerative fiber constituting the eco-friendly structured body according to one aspect of the present disclosure was evaluated using the same method as in Example 1 above, and the results are shown in Table 6. In this case, the eco-friendly structured body used cellulose as the natural fiber and PET as the regenerative fiber, and the weight ratio of the natural fiber to the regenerative fiber was approximately 40:60, and the average thickness of the fibers was 20 μm, and 15 grams of the cosmetic composition of Example 1 above was carried.

TABLE 6

| | Compar- | Compar- | Compar- | | | | | | Compar- | Compar- |
|---|---|---|---|---|---|---|---|---|---|---|
| | ative | ative | ative | | | | | | ative | ative |
| | exam- | exam- | exam- | Exam- | Exam- | Exam- | Exam- | Exam- | exam- | exam- |
| | ple | ple | ple | ple | ple | ple | ple | ple | ple | ple |
| | 4-1 | 4-2 | 4-3 | 4-1 | 4-2 | 4-3 | 4-4 | 4-5 | 4-4 | 4-5 |
| Fiber | 10 | 20 | 30 | 40 | 50 | 60 | 70 | 80 | 90 | 100 |
| length | | | | | | | | | | |
| (mm) | | | | | | | | | | |
| Stability | Unstable | Unstable | Unstable | Stable | Stable | Stable | Stable | Stable | Unstable | Unstable |

As shown in Table 6 above, when the average length of the fibers constituting the structured body was less than 40 mm (Comparative examples 4-1 to 4-3), fluff formed on the surface of the fibers, which reduced the stability. In addition, when the average length of the fibers was greater than 80 mm (Comparative examples 4-4 and 4-5), the oil phase component of the cosmetic composition carried inside the structured body moved along the fibers, causing the oil phase component to be separated and not suitably carrying the liquid phase of the cosmetic composition.

In contrast, when the average length of the fibers was 40 to 80 mm (Examples 4-1 to 4-3), the oil phase of the cosmetic composition was not separated, so the cosmetic composition was stably contained in the structured body (carrier), and it was confirmed that the cosmetic composition had excellent stability by not forming fluff even when used for a long time.

### [Example 6] Comparison of Discharge Capability and Sense of Use According to Fiber Shape

The discharge capability and sense of use according to the shapes of the natural fiber and the regenerative fiber constituting the eco-friendly structured body according to one aspect of the present disclosure was evaluated, and the results are shown in Table 7. In this case, the eco-friendly structured body used cellulose as the natural fiber and PET as the regenerative fiber. In addition, the weight ratio of the natural fiber to the regenerative fiber is 40:60, the average thickness of the fibers is approximately 20 μm, the average length of the fibers is approximately 45 mm, and 15 grams of the cosmetic composition of Example 1 above is carried.

The discharge capability was evaluated in the same method as in Experimental example 2 above, and the sense of use was evaluated in the same method as in Experimental example 4 above.

TABLE 7

| | Comparative example 5 | Example 5 |
|---|---|---|
| Shape of | Linear | Corrugated (Coil) |
| regenerative fiber | [symbol] | [symbol] |
| Discharge capability | ◯ | ⊚ |
| Sense of use | ◯ | ⊚ |

As shown in Table 7 above, it was confirmed that when the fibers constituting the structured body are formed in a corrugated form with unevenness, bumps, or twists (Example 5) than when the fibers are formed in a linear form (Comparative example 5), the elasticity increases and the cosmetic composition carried inside is uniformly discharged in a more suitable amount, and the sense of use is also excellent.

Accordingly, the eco-friendly structured body according to one aspect of the present disclosure has an effect of being capable of carrying the cosmetic composition therein, of discharging the carried cosmetic composition while having elasticity, of stably carrying the cosmetic composition while remaining in a usable state during the period of use after being manufactured, of being naturally decomposed thereafter, of having eco-friendly properties, and of having excellent stability such that the oil phase component is not separated even when the cosmetic composition including the oil phase and water phase components is carried therein.

The present disclosure may provide the following embodiments as examples.

A first embodiment may provide an eco-friendly structured body including a natural fiber and a regenerative fiber, and having one or more of characteristics of (A) to (C), in which: (A) an average length of one or more of those selected from the group consisting of the natural fiber and the regenerative fiber is 40 to 80 mm; (B) a weight ratio of the natural fiber to the regenerative fiber is 85:15 to 20:80; and (C) an average thickness of one or more of those selected from the group consisting of the natural fiber and the regenerative fiber is 15 to 25 μm.

A second embodiment may provide the eco-friendly structured body according to the first embodiment, in which the natural fiber is one or more of those selected from the group consisting of a cellulose-based fiber and a protein-based fiber.

A third embodiment may provide the eco-friendly structured body according to the first or second embodiment, in which the regenerative fiber is one or more of those selected from the group consisting of polyester (PET), polyethylene (PE), polypropylene (PP), thermoplastic elastomer (TPE), thermoplastic polyurethane (TPU), and natural pulp fiber.

A fourth embodiment may provide the eco-friendly structured body according to any one or more of the first to third embodiments, in which the structured body is a structure in which the natural fiber and the regenerative fiber are twisted.

A fifth embodiment may provide the eco-friendly structured body according to one or more of the first to fourth embodiments, in which at least a portion of the surface of one or more of those selected from the group consisting of the natural fiber and the regenerative fiber is formed with unevenness, bumps, or twists.

A sixth embodiment may provide the eco-friendly structured body according to one or more of the first to fifth embodiments, in which the structured body includes a fiber having a ratio of a longest diameter to a shortest diameter of a cross-section of one or more of those selected from the group consisting of the natural fiber and the regenerative fiber exceeding 1.

A seventh embodiment may provide the eco-friendly structured body according to one or more of the first to sixth embodiments, in which the structured body has a density of 0.015 to 0.032 g/cm³.

An eighth embodiment may provide the eco-friendly structured body according to one or more of the first to seventh embodiments, in which the structured body has a thickness of 0.1 to 10 mm.

A ninth embodiment may provide the eco-friendly structured body according to one or more of the first to eighth embodiments, in which the structured body is for carrying a cosmetic composition.

A tenth embodiment may provide the eco-friendly structured body according to the ninth embodiment, in which the structured body has a structure including at least a plurality of storage spaces in which a portion of the carried cosmetic composition is collected.

An eleventh embodiment may provide the eco-friendly structured body according to the ninth or tenth embodiment, in which the cosmetic composition is a liquid phase composition.

A twelfth embodiment may provide the eco-friendly structured body according to one or more of the ninth to eleventh embodiments, in which the cosmetic composition has a viscosity of 5,000 to 20,000 centi poise (cps).

A thirteenth embodiment may provide the eco-friendly structured body according to one or more of the ninth to twelfth embodiments, in which the structured body is for use by applying the cosmetic composition carried in the structured body to an application tool.

A fourteenth embodiment may provide a cosmetic product including the eco-friendly structured body according to one or more of the first to thirteenth embodiments, and a cosmetic composition carried in the eco-friendly structured body.

A fifteenth embodiment may provide the cosmetic product of the fourteenth embodiment, in which the cosmetic product further includes an application tool.

What is claimed is:

1. An eco-friendly structured body comprising a natural fiber and a regenerative fiber, and having one or more of characteristics of (A) to (C), wherein:
(A) an average length of one or more of those selected from the group consisting of the natural fiber and the regenerative fiber is 40 to 80 mm;
(B) a weight ratio of the natural fiber to the regenerative fiber is 85:15 to 20:80; and
(C) an average thickness of one or more of those selected from the group consisting of the natural fiber and the regenerative fiber is 15 to 25 μm.

2. The eco-friendly structured body of claim 1, wherein the natural fiber is one or more of those selected from the group consisting of a cellulose-based fiber and a protein-based fiber.

3. The eco-friendly structured body of claim 1, wherein the regenerative fiber is one or more of those selected from the group consisting of rayon, polyester (PET), polyethylene (PE), polypropylene (PP), thermoplastic elastomer (TPE), thermoplastic polyurethane (TPU), natural pulp fiber, nylon, and acetate.

4. The eco-friendly structured body of claim 1, wherein the structured body is a structured body in which the natural fiber and the regenerative fiber are twisted.

5. The eco-friendly structured body of claim 1, wherein at least a portion of a surface of one or more of those selected from the group consisting of the natural fiber and the regenerative fiber is formed with unevenness, bumps, or twists.

6. The eco-friendly structured body of claim 1, wherein the structured body includes a fiber having a ratio of a longest diameter to a shortest diameter of a cross section of one or more of those selected from the group consisting of the natural fiber and the regenerative fiber exceeding 1.

7. The eco-friendly structured body of claim 1, wherein the structured body has a density of 0.015 to 0.032 g/cm³.

8. The eco-friendly structured body of claim 1, wherein the structured body has a thickness of 0.1 to 10 mm.

9. The eco-friendly structured body of claim 1, wherein the structured body is for carrying a cosmetic composition.

10. The eco-friendly structured body of claim 9, wherein the structured body includes a plurality of storage spaces in which a portion of the carried cosmetic composition is collected.

11. The eco-friendly structured body of claim 9, wherein the cosmetic composition is a liquid phase composition.

12. The eco-friendly structured body of claim 9, wherein the cosmetic composition has a viscosity of 5,000 to 20,000 centi poise (cps).

13. The eco-friendly structured body of claim 9, wherein the structured body is for use by applying the cosmetic composition carried in the structured body to an application tool.

14. A cosmetic product comprising:
the eco-friendly structured body according to claim 1; and
a cosmetic composition carried in the eco-friendly structured body.

15. The cosmetic product of claim 14, wherein the cosmetic product further comprises an application tool.
